(19) United States
(12) Patent Application Publication
(10) Pub. No.: US 2005/0098466 A1
(43) Pub. Date: May 12, 2005

(54) Feminine protection product disposal pouch

(76) Inventor: Kathleen A. Thomas, Macomb Township, MI (US)

Correspondence Address: GREGORY T. ZALECKI, 12900 HALL ROAD, SUITE 400, STERLING HEIGHTS, MI, 48313, US

(21) Appl. No.: 10/705,008

(22) Filed: Nov. 10, 2003

(51) Int. Cl. A61L 15/00

(52) U.S. Cl. 206/440; 206/438; 206/205

(57) ABSTRACT

A feminine protection product disposal pouch comprising a flexible leakproof pouch. The pouch is sized and shaped to compactly contain a used feminine protection product. The pouch is formed by two opaque decorative walls which camouflage the purpose of the pouch. An adhesive covered flap, covered by an adhesive cover, extends from one of the walls and can be folded over the opening of the pouch to seal it after the cover is removed. The pouch contains odor absorbing material and scented material to disguise and neutralize any odor of the pouch contents. The pouch can be sized and shaped to allow the discrete disposal of other personal protection products.

### BACKGROUND

Women of childbearing age regularly use feminine protection products. As used herein, the term feminine protection products means sanitary pads and tampons. When a woman is using a feminine protection product regular replacement and disposal of used feminine protection products is required.

A used feminine protection product should not be flushed down a toilet. It may cause jamming within the plumbing and require the use of a plunger or the use of a plumber's services. A dry method of disposal is preferred. Typically, such a method involves disposing of the used feminine protection product within a garbage can or a wastebasket.

Dry disposal within a garbage can or a wastebasket has several characteristics which are undesirable to many women. The used feminine protection product may have an odor. It is considered unsightly. Therefore, many women prefer not to dispose of a used feminine protection product at the home of another or at a public restroom. They also prefer not to carry a used feminine protection product with them because it is considered unsightly, may emit an odor and it would be embarrassing if it were identified by another person. If the used product could be carried within a woman's purse, not be identifiable and not emit an odor, many women would prefer to carry the product with them until they were able to dispose of it at their residence. It would also be less embarrassing for a woman to dispose of a used feminine protection product at a public restroom or at someone else's residence if the identity of and the odor of the used feminine protection product could be camouflaged.

Information relevant to attempts to address these problems can be found in U.S. Pat. Nos. 6,402,727, 6,231,234, 5,647,100 and 4,857,066. However, each one of these references suffers from one or more of the following disadvantages: it is not discrete (i.e. its purpose is not camouflaged); it does not conceal or absorb the odor of a used feminine protection product; it is not easy to use; it is not able to be manufactured at a low cost; it is not leakproof; or, it will not conveniently fit within a woman's purse.

What is needed is a small leakproof pouch which would fit within a woman's purse. The pouch should be capable of securely sealing a used feminine protection product within it. It should be capable of camouflaging its purpose. It should be odor absorbing. Preferably, it should emit a desirable scent. It should be easy to use. It should be able to be manufactured at a low cost.

### SUMMARY

The present invention is directed to a feminine protection product disposal pouch that satisfies these needs. The feminine protection product disposal pouch comprises a flexible leakproof pouch. It is sized and shaped to compactly contain a used feminine protection product. Thus, it is preferably sized and shaped to be no larger than necessary to contain a used feminine protection product. This will permit the pouch to be conveniently stored within a woman's purse. The pouch is comprised of two walls. The walls are opaque. Thus, a person will be unable to see a used feminine protection product sealed within the pouch. The walls are also decorative. For example, the walls may have a flowered design imprinted upon them. This disguises the purpose of the pouch. The pouch has an opening at one end. This permits a used feminine protection product to be inserted into the pouch. The pouch also has a flap. The flap has a sealing side and an exposed side. The flap is sized and positioned such that it may be used to cover and seal the opening of the pouch.

Preferably, the feminine protection product disposal pouch further comprises an odor absorbing material. The odor absorbing material is placed within the pouch. There, it is capable of absorbing the odor of an article placed within the pouch. The odor absorbing material may be sprayed onto the inside surfaces of the pouch walls. It may also be molded into the walls of the pouch. Charcoal or sodium bicarbonate may be used as the odor absorbing material.

A scented material may also be placed within the pouch. The scented material may be a perfume. The scented material within the pouch will disguise the odor of an article placed within the pouch. The scented material may be sprayed onto the inside surfaces of the pouch walls. It may also be molded into the walls of the pouch.

Preferably, the sealing side of the flap is covered with an adhesive and the adhesive is covered with a removable nonstick cover. The nonstick cover prevents the adhesive from adhering to anything prior to the intended use of the pouch. When the pouch is used to contain a used feminine protection product the adhesive will cause the flap to be firmly secured over the opening of the pouch.

### DRAWINGS

These and other features, aspects, and advantages of the present invention will become better understood with regard to the following description, appended claims, and accompanying drawings where:

FIG. 1 is a perspective view of a feminine protection product disposal pouch showing the adhesive cover on the pouch partially removed and exposing a portion of the flap adhesive;

FIG. 2 is a cross-sectional view of the feminine protection product disposal pouch of FIG. 1 showing scented material and odor absorbing material sprayed onto the inside surfaces of the walls of the pouch, a feminine protection product inserted into the pouch, the flap covering the opening of the pouch and uncovered flap adhesive about to contact the pouch wall to seal the pouch;

FIG. 3 is a cross-sectional view of the feminine protection product disposal pouch of FIG. 1 showing an odor absorbing material or a scented material within the pouch; and

FIG. 4 is a top view of an alternate embodiment of a feminine protection product disposal pouch showing odor absorbing material and scented material molded into a wall of the pouch.

### DESCRIPTION

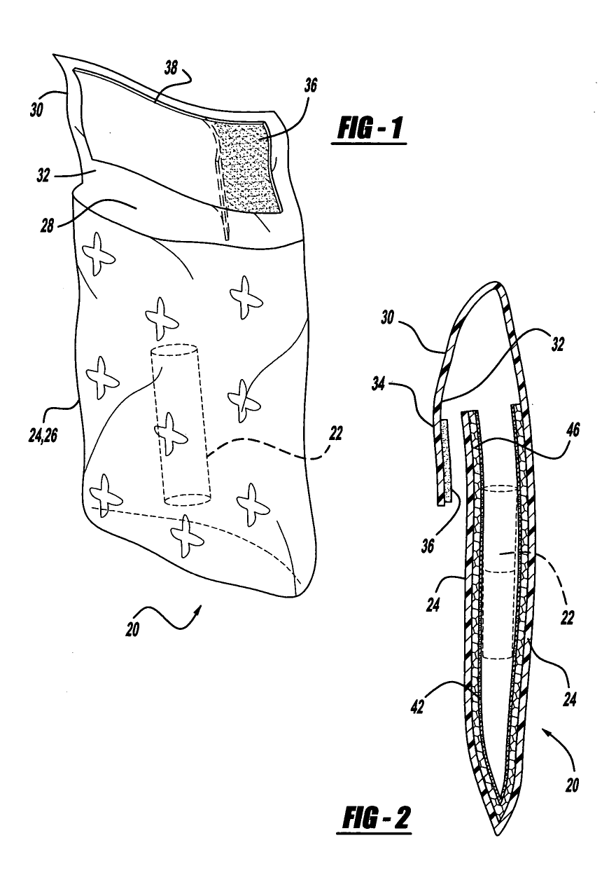

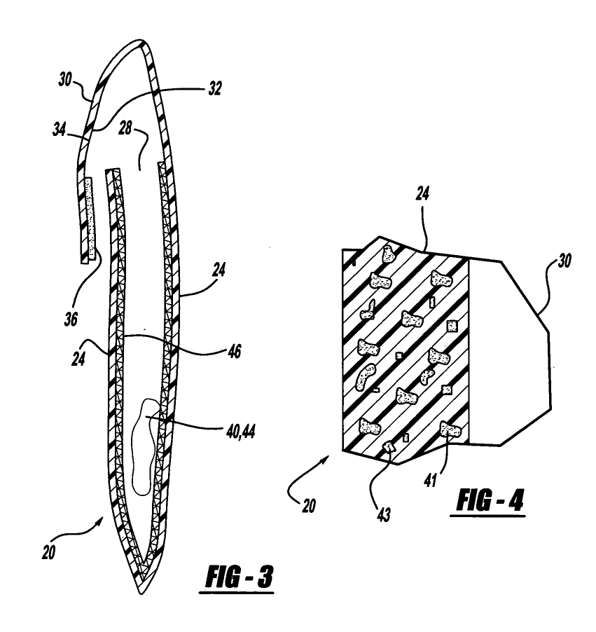

FIG. 1 shows the preferred embodiment of a feminine protection product disposal pouch 20. It may be constructed from many flexible and leakproof materials. These include polyethylene, latex, cellophane and rubber. The preferred material is polyethylene. Polyethylene provides the necessary leakproof and flexibility features for the pouch 20. Because the pouch 20 is leakproof any liquid or semiliquid material placed within the pouch 20 will be retained within the pouch 20. The pouch 20 is formed from two opaque walls 24. The opacity of the walls 24 inhibits the ability of a person to see into the pouch 20. Preferably, the walls 24 are opaque decorative walls 26. For example, the walls 24 may have a flowered design imprinted upon them. This camouflages the purpose of the pouch 20. The pouch 20 has an opening 28 at one end for permitting the insertion of a used feminine protection product 22. The pouch 20 is sized and shaped to compactly contain a used feminine protection product 22. It should be sized and shaped to be no larger than necessary to contain a used feminine protection product 22.

A flap 30 is extended from one of the walls 24 such that it may be folded to cover the opening 28 of the pouch 20. The flap 30 has two sides. One side is the exposed side 34. The other side is the sealing side 32. An adhesive 36 is applied to a portion of the sealing side 32 of the flap 30. The adhesive 36 is covered by a removable nonstick cover 38. The adhesive 36 must have characteristics which permit it to retain its adhesive properties after the nonstick cover 38 is applied to the adhesive 36 and removed from the adhesive 36. Further, the adhesive 36 must have a property which causes the flap 30 to be able to be securely closed over the opening 28 of the pouch 20 and result in a leakproof sealing of the pouch 20.

An odor absorbing material 40 is positioned within the pouch 20. The odor absorbing material 40 may be charcoal or sodium bicarbonate. Other well known odor absorbing materials 40 may also be used. Preferably, the odor absorbing material 40 is affixed to one of the walls 24 of the pouch 20. An adhesive can be used to affix the odor absorbing material to the wall 24. Alternatively, the odor absorbing material 40, 42 may be sprayed onto the wall 24 surfaces within the pouch 20, as shown in FIG. 2, or, it may be molded into the walls 24, as shown in FIG. 4.

In the preferred embodiment of the feminine protection product disposal pouch 20 a scented material 44 is placed within the pouch 20. The scented material 44 will camouflage any odor emitted by the used feminine protection product 22. Preferably, the scented material 44 is a perfume affixed to one of the walls 24 of the pouch 20. The perfume may be embedded within a substrate such as cotton and the cotton affixed to the surface of a wall 24 forming the inside of the pouch 20. Alternatively, the scented material 44, 46 may be sprayed onto the wall 24 surfaces within the pouch 20, as shown in FIG. 2, or, it may be molded into the walls 24, as shown in FIG. 4. FIG. 4 shows an odor absorbing material 41 molded into the walls 24 and a scented material 43 molded into the walls.

To use the feminine protection product disposal pouch 20 a used feminine protection product 22 is placed into the pouch 20, as shown in FIG. 1. The user removes the nonstick cover 38, exposing the adhesive 36. The flap 30 is then tightly folded over the opening 28 of the pouch 20, as shown in FIG. 2. The adhesive end of the flap 30 is then pressed onto the opposing pouch wall 24, thereby causing the used feminine protection product 22 to be sealed within a leakproof opaque decorative pouch. The sealed pouch 20 can be placed within the user's purse or other personal container and subsequently discarded.

A feminine personal protection product 22 is a specific type of a personal protection product. Personal protection products are intended for personal hygiene. They include diapers for both infants and adults. Typically, a user of a personal protection product desires to be able to discreetly dispose of the personal protection product, just as a user of a feminine personal protection product 22 desires to be able to discreetly dispose of the feminine personal protection product 22.

A feminine protection product disposal pouch 20 can be easily modified to serve as a personal protection product disposal pouch. This is accomplished by sizing and shaping the pouch to compactly contain a personal protection product. Thus, the personal protection product disposal pouch would be sized and shaped large enough to contain a used personal protection product, but no larger. This optimizes the ability of a pouch user to discreetly dispose of a used personal protection product.

The drawings and description above show that the pouch 20 is closed by folding a flap 30 over the opening 28 of the pouch 20. This invention is not limited to pouches which close by having a flap folded over their respective openings. A feminine protection product disposal pouch 20 and a personal protection product disposal pouch can have their openings sealed by other well-known methods and devices which provide leakproof closures for flexible leakproof pouches. These methods and devices include zippered closures and groove and rib closures. In a groove and rib type of closure the periphery at the opening of one pouch wall is comprised of a rib. The periphery at the opening of the other pouch wall is comprised of a groove. The pouch is sealed by manually pressing the rib into the groove. The trade name for bags being currently sold which contain a rib and a groove for closure is ZIPLOCK.

1. A feminine protection product disposal pouch comprising a flexible leakproof pouch sized and shaped to compactly contain a used feminine protection product, said disposal pouch further comprising two opaque decorative walls forming the pouch, the pouch having an opening for inserting the used feminine protection product and a flap having a sealing side and an exposed side for covering and sealing the opening of the pouch.

2. The feminine protection product disposal pouch of claim 1, further comprising an odor absorbing material placed within the pouch for absorbing odor.

3. The feminine protection product disposal pouch of claim 2, wherein the odor absorbing material is sprayed onto the wall surfaces within the pouch.

4. The feminine protection product disposal pouch of claim 2, wherein the odor absorbing material is charcoal.

5. The feminine protection product disposal pouch of claim 2, wherein the odor absorbing material is sodium bicarbonate.

6. The feminine protection product disposal pouch of claim 3, wherein the odor absorbing material is charcoal.

7. The feminine protection product disposal pouch of claim 3, wherein the odor absorbing material is sodium bicarbonate.

8. The feminine protection product disposal pouch of claim 2, wherein the odor absorbing material is molded into the walls of the pouch.

9. The feminine protection product disposal pouch of claim 1, further comprising a scented material placed within the pouch.

10. The feminine protection product disposal pouch of claim 9, wherein the scented material is sprayed onto the wall surfaces within the pouch.

11. The feminine protection product disposal pouch of claim 9, wherein the scented material is molded into the walls of the pouch.

12. The feminine protection product disposal pouch of claim 1, wherein a portion of the sealing side of the flap is covered with an adhesive and the adhesive is covered with a removable nonstick cover.

13. A feminine protection product disposal pouch comprising a flexible leakproof pouch sized and shaped to compactly contain a used feminine protection product, said disposal pouch further comprising two opaque decorative walls forming the pouch for camouflaging the purpose of the pouch, the pouch having an opening for inserting the used feminine protection product and a flap having a sealing side and an exposed side for covering and sealing the opening of the pouch, a portion of said sealing side of the flap being covered with an adhesive and said adhesive being covered with a removable nonstick cover, said pouch having an odor absorbing material within it for absorbing odor.

14. The feminine protection product disposal pouch of claim 13, wherein the odor absorbing material is sprayed onto the wall surfaces within the pouch.

15. The feminine protection product disposal pouch of claim 13, wherein the odor absorbing material is charcoal.

16. The feminine protection product disposal pouch of claim 13, wherein the odor absorbing material is sodium bicarbonate.

17. The feminine protection product disposal pouch of claim 13, wherein the odor absorbing material is molded into the walls of the pouch.

18. The feminine protection product disposal pouch of claim 13, further comprising a scented material placed within the pouch.

19. The feminine protection product disposal pouch of claim 13, wherein the scented material is sprayed onto the wall surfaces within the pouch.

20. The feminine protection product disposal pouch of claim 13, wherein the scented material is molded into the walls of the pouch.

21. A feminine protection product disposal pouch comprising a flexible leakproof pouch sized and shaped to compactly contain a used feminine protection product, said disposal pouch further comprising two opaque decorative walls forming the pouch for camouflaging the purpose of the pouch, the pouch having an opening for inserting the used feminine protection product and a flap having a sealing side and an exposed side for covering and sealing the opening of the pouch, a portion of said sealing side of the flap being covered with an adhesive and said adhesive being covered with a removable nonstick cover and said pouch having an odor absorbing material molded into its walls.

22. A feminine protection product disposal pouch comprising a flexible leakproof pouch sized and shaped to compactly contain a used feminine protection product, said disposal pouch further comprising two opaque decorative walls forming the pouch for camouflaging the purpose of the pouch, the pouch having an opening for inserting the used feminine protection product and a flap having a sealing side and an exposed side for covering and sealing the opening of the pouch, a portion of said sealing side of the flap being covered with an adhesive and said adhesive being covered with a removable nonstick cover, said pouch having an odor absorbing material molded into its walls and said pouch having a scented material molded into its walls.

23. A personal protection product disposal pouch comprising a flexible leakproof pouch sized and shaped to compactly contain a used personal protection product, said disposal pouch further comprising two opaque decorative walls forming the pouch for camouflaging the purpose of the pouch, the pouch having an opening for inserting the used personal protection product and a flap having a sealing side and an exposed side for covering and sealing the opening of the pouch, a portion of said sealing side of the flap being covered with an adhesive and said adhesive being covered with a removable nonstick cover, said pouch having an odor absorbing material within it for absorbing odor.

24. The personal protection product disposal pouch of claim 23, wherein the odor absorbing material is sprayed onto the wall surfaces within the pouch.

25. The personal protection product disposal pouch of claim 23, wherein the odor absorbing material is charcoal.

26. The personal protection product disposal pouch of claim 23, wherein the odor absorbing material is sodium bicarbonate.

27. The personal protection product disposal pouch of claim 23, wherein the odor absorbing material is molded into the walls of the pouch.

28. The personal protection product disposal pouch of claim 23, further comprising a scented material placed within the pouch.

29. The personal protection product disposal pouch of claim 23, wherein the scented material is sprayed onto the wall surfaces within the pouch.

30. The personal protection product disposal pouch of claim 23, wherein the scented material is molded into the walls of the pouch.
